(19) United States
(12) Patent Application Publication
(10) Pub. No.: US 2016/0287353 A1
(43) Pub. Date: Oct. 6, 2016

(54) Mandibular Advancement Positioning Bite Blocks and Positioning System

(71) Applicant: Joseph G. Mirci, Salt Lake City, UT (US)

(72) Inventor: Joseph G. Mirci, Salt Lake City, UT (US)

(21) Appl. No.: 14/674,931

(22) Filed: Mar. 31, 2015

(51) Int. Cl. A61C 5/00; A61C 9/00; A61B 5/087; A61B 5/097; A61B 5/00; A61F 5/56

(52) U.S. Cl. CPC A61C 5/007; A61B 5/4542; A61F 5/566; A61B 5/4818; A61B 5/087; A61B 5/097; A61C 9/00

(57) ABSTRACT

The invention is directed to improved jigs or mandibular positioning bite blocks for positioning the mandible relative to upper jaw for treatment of obstructive sleep apnea and/or snoring, as well as a system for mandibular advancement positioning. The invention provides for controlled variations for both (1) forward (protruded) and back positions in the anterior plane of the mandible relative to the jaw and (2) bite opening positions in the vertical plane.

### BACKGROUND OF THE INVENTION

1. The Field of the Invention

The present invention relates to a bite block and a mandibular advancement positioning system. More particularly, the present invention relates to a bite block for diagnostic testing and to facilitate fabrication of related dental impressions and intra-oral dental appliances for the treatment of obstructive sleep apnea and/or snoring.

2. The Relevant Technology

Snoring and obstructive sleep apnea are typically caused by complete or partial obstruction of an individual's pharyngeal airway during sleep. Usually, airway obstruction results from the apposition of the rear portion of the tongue or soft palate with the posterior pharyngeal wall.

Obstructive sleep apnea is a potentially lethal disorder in which breathing stops during sleep for 10 seconds or more, sometimes up to 300 times per night. Snoring occurs when the pharyngeal airway is partially obstructed, resulting in vibration of the oral tissues during respiration. These sleep disorders tend to become more severe as patients grow older, likely as a result of a progressive loss of muscle tone in the patient's throat and oral tissues.

Habitual snoring and sleep apnea have been associated with other potentially serious medical conditions, such as hypertension, ischemic heart disease and strokes. Accordingly, early diagnosis and treatment is recommended.

One surgical approach, known as uvulopalatopharyngoplasty, involves removal of a portion of the soft palate to prevent closure of the pharyngeal airway during sleep. This operation, however, is not always effective and may result in undesirable complications, such as nasal regurgitation.

A wide variety of non-surgical approaches for treating sleep disorders have been proposed including the use of oral cavity appliances. It has been previously recognized that movement of the mandible (lower jaw) forward relative to the maxilla (Upper jaw) can eliminate or reduce sleep apnea and snoring symptoms by causing the pharyngeal air passage to remain open.

Several intra-oral dental appliances have been developed which the user wears at night to fix the mandible in an anterior, protruded (i.e. forward) position. Such dental appliances essentially consist of acrylic or elastomeric bite blocks, similar to orthodontic retainers or athletic mouth guards, which are custom-fitted to the user's upper and lower teeth and which may be adjusted to vary the degree of anterior protrusion and/or bite opening.

In diagnostic testing various jigs or bite blocks may be used to determine the optimal position of the mandible relative to the upper jaw in order to maximize airflow. For example, a George Gauge is a standard instrument that can be used to vary and replicate the bite registration for mandibular advancement appliances. While the George Gauge commonly allows the mandibular advancement to be varied, it does not provide a convenient mechanism for also varying the bite opening in the vertical plane. Another disadvantage with the standard George Gauge is that it cannot be used to position a patient's jaw during diagnostic flow testing using a pharyngometer.

Another product found in the prior art is a kit produced by a company by the name of Airway Metrics (“AWM”), which consists of a set of mandibular positioning bite blocks and a set of vertical titration keys. An exemplary illustration of one example of an AWM bite block is shown in FIG. 1. The bite block 1 illustrated in FIG. 1 is intended to be placed between a patient's upper and lower central incisors, with the first or posterior end 2 extending behind the patient's teeth and the second or anterior end 3 extending in front of the patient's teeth. A single notch 4 is provided in the lower edge to be engaged by a portion of the patient's lower central incisors, and one or more notches, such as notches 5a-5c, may be provided in the upper edge to be engaged by a portion of the patient's upper central incisors. The positions of upper notches 5a-5c relative to lower notch 4 determines the position of the patient's upper and tower jaws relative to one another. For example, with the patient's lower incisor positioned in lower notch 4, the patient's upper incisor can be selectively positioned in upper notch 5a (so that the patient's lower jaw is positioned in an “advanced” position relative to the upper jaw), or can be selectively positioned in upper notch 5b (so that the patient's upper and lower jaws are relatively aligned), or can be selectively positioned in upper notch 5c (so that the patient's upper jaw is positioned forward of the lower jaw). In addition, the distance separating lower notch 4 from upper notches 5a-5c determines the size of the bite opening.

One of the disadvantages with the AWM bite blocks is that the notches provided to be engaged by the patient's teeth are not deep enough to stabilize the jaw during fitting and testing. Consequently, unless used with a bite fork and/or handle, the bite blocks can pivot or rock back and forth, allowing movement of the mandible relative to the upper jaw. While the use of a bite fork and/or handle does help to stabilize the AWM bite blocks, such added components interfere with the use of a pharyngometer for diagnostic airflow testing. Another disadvantage with the AWM bite blocks is that each bite block provides multiple notches, which can complicate the process of positioning the patient's teeth in the desired notches and keeping track of which positions result in improved airflow.

Thus, what is needed is an improved bite block that can securely, stably and repeatably position a patient's mandible in a desired position relative to the upper jaw, that can selectively, precisely and securely position the upper and lower jaws relative to one another in both the horizontal and the vertical planes, and yet is small enough to tit inside the primary lumen of a pharyngometer to facilitate use during diagnostic airflow testing.

### BRIEF SUMMARY OF THE INVENTION

The invention is directed to improved jigs or mandibular positioning bite blocks for positioning the mandible relative to upper jaw for treatment of snoring and/or obstructive sleep apnea. The invention provides means for controlled variations for both (1) forward (protruded) and rearward positions in the horizontal plane of the mandible relative to the upper jaw and (2) bite opening positions in the vertical plane. Bite notches/indentations are provided on the upper and lower surfaces of the bite blocks and are configured to receive a portion of a patient's upper and tower central incisors, respectively. Such bite notches/indentations are sized and configured such that, when the patient bites down, the bite block supports and secures the mandible and upper jaw in a stable position relative to one another (i.e., the patient's jaws, as ell as the bite block, are secure, stable and stationary). In other words, the bite blocks do not require the attachment of any other components, such as a handle, bite fork or other mechanical element, to stabilize the patient's jaw or the bite block itself.

The bite blocks also preferably provide a small form factor. The small size facilitates airflow testing, using a pharyngometer to find the optimal relative positioning of the mandible relative to the upper jaw for maximum airflow and/or maximum reduction in snoring and/or obstructive sleep apnea. The compact form factor also enables the device to guide, and stably maintain, the mandible relative to the upper jaw into the optimal relative position during the process of making dental/bite impressions, which can then be used to fabricate a suitable intra-oral appliance that can be worn by the patient to help reduce or eliminate snoring and/or obstructive steep apnea symptoms.

The position of the upper bite notch/indentation relative to the position of the lower bite notch/indentation controls the position of the mandible relative to the upper jaw in the horizontal plane, and can be varied in predetermined increments (e.g., −2 mm, −1 mm, 0, +1 mm, +2 mm, +3 mm, +4 mm, etc.). Similarly, the distance between the apex of the upper bite notch/indentation and the apex of the lower bite notch/indentation determines the size of the bite opening in the vertical plane, which can also be varied in predetermined increments (e.g., 4 mm, 6 mm, etc.).

The improved bite block is also able to stably maintain the patient's jaw in the desired position during diagnostic airflow testing using a pharyngometer. Once the optimal position for maximizing the patient's airflow is determined, the improved bite blocks is able to guide the patient's jaw into the desired position, and then stably maintain the patient's jaw in that desired position, during fabrication of dental impressions.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates one example of a prior art bite block.

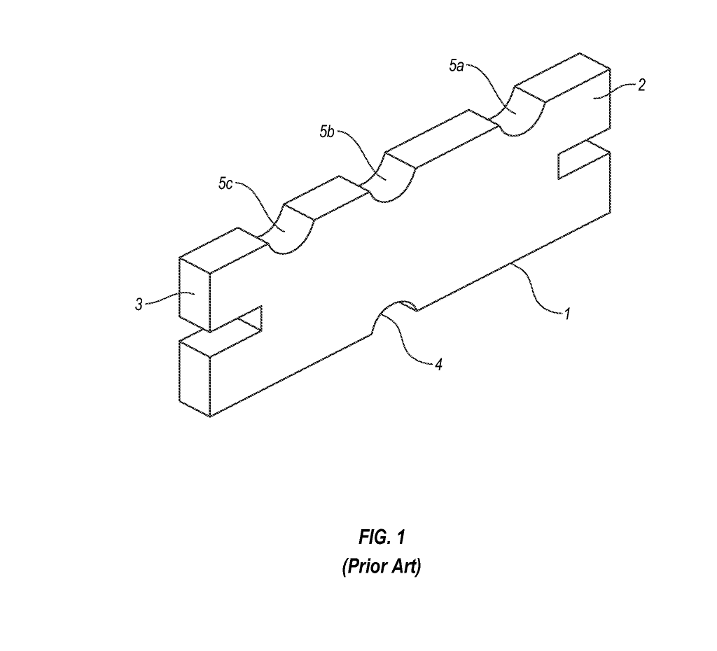

FIG. 2 is a perspective view of one embodiment of a bite block of the present invention.

FIG. 3 is another perspective view of the bite block of FIG. 2.

FIG. 4 is an enlarged plan view of the bite block of FIG. 2.

FIG. 5 schematically illustrates placement of a bite block between the teeth of a patient.

FIG. 6 illustrates a kit container for housing a plurality of bite blocks with various combinations of mandible positions and bite opening positions.

### DETAILED DESCRIPTION OF THE PREFERRED EMBODIMENTS

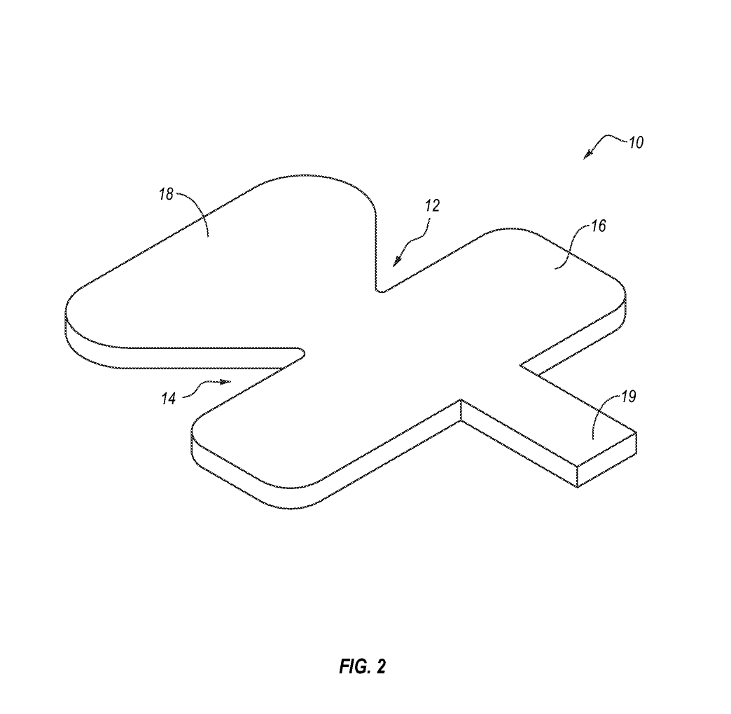

One embodiment of a bite block 10 in accordance with the present invention is illustrated in FIGS. 2-4. It should be understood that the embodiment illustrated in, and described in relation to FIGS. 2-4 merely represents one representative example of the invention and that, in practice, a plurality of bite blocks 10 are to be provided and used in practice, each bite block 10 providing a different combination of dimensions for positioning a patient's lower jaw relative to the upper jaw in both horizontal and vertical directions. Thus, the invention contemplates a plurality of bite blocks of varying sizes and dimensions that are sized, shaped, and configured to function as more fully described below.

As used herein: “superior” refers to the direction toward the top of the oral cavity, “inferior” refers to the direction toward the bottom of the oral cavity; “anterior” refers to the direction toward the front of the oral cavity or tips; “posterior” refers to the direction toward the back of the oral cavity and airway; “horizontal” or “longitudinal” refers to the direction generally in the forward and/or backward direction in the oral cavity; and “vertical” refers to the direction generally in the up and/or down direction in the oral cavity.

In one embodiment, the bite blocks may be configured generally as illustrated FIG. 2. Bite Block 10 may have an upper notch 12, a lower notch 14, an anterior portion 16 located anterior of notches 12 and 14, a posterior portion 18 located posterior of notches 12 and 14, and a stem 19 located anterior of anterior portion 16. As discussed in additional detail below, bite block 10 is configured to receive a portion of a patient's upper central incisors in notch 12 and to receive a portion of the patient's lower central incisors in notch 14, so that anterior portion 16 is positioned anterior to, or in front of, the patient's central incisors and posterior portion 18 is positioned posterior to, or behind, the patient's central incisors. Stem 19 may be provided to facilitate manipulation and placement of bite block 10 by a dental professional or the patient.

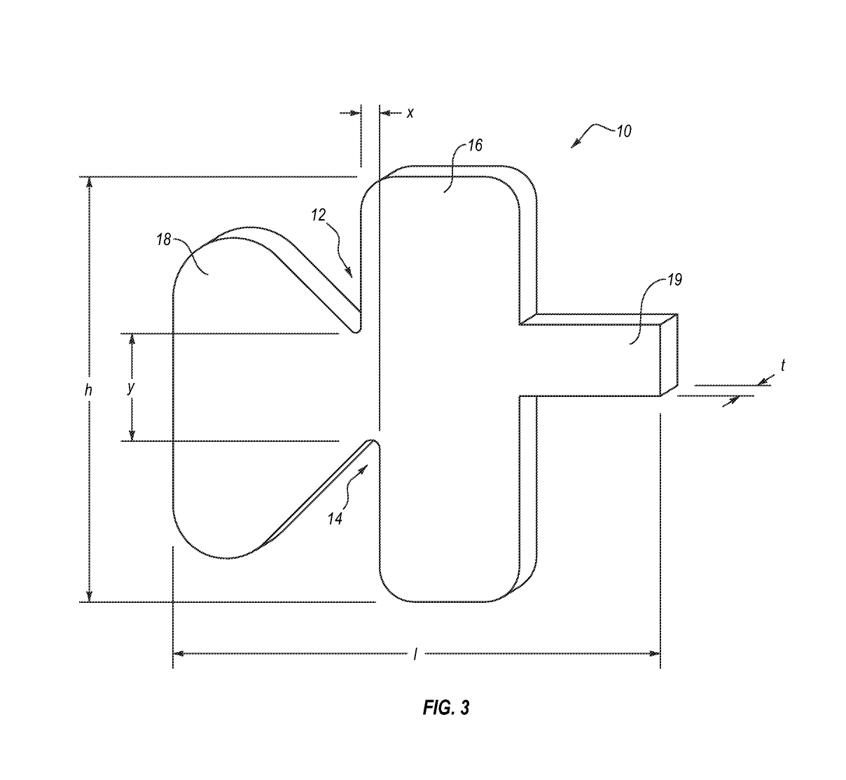

Referring to FIG. 3, bite block 10 preferably has a small form factor, to facilitate its use to position a patient's mandible relative to the upper jaw without significantly interfering with diagnostic airflow testing and/or the making of dental impressions. For adult patients, the overall height (h) of bite block 10 may be in the range of from approximately 15 mm to approximately 25 mm, and may preferably be approximately 20 mm. For adult patients, the overall length (l) of bite block 10 may be in the range of from approximately 20 mm to approximately 30 mm, and may preferably be approximately 25 mm. And, for adult patients, the transverse thickness (t) of bite block 10 may be in the range of from approximately 2 mm to approximately 4 mm, and may preferably be approximately 3 mm. It will be appreciated that bite blocks 10 may also be appropriately reduced in size, as necessary, or appropriate, for use with smaller and/or younger patients.

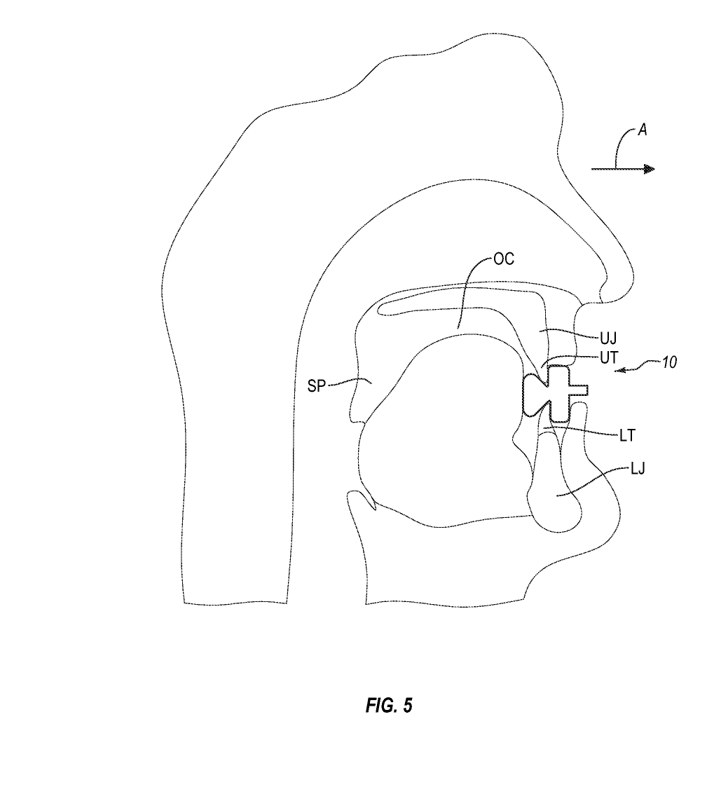

Notwithstanding its small form factor, bite block 10 is configured to stably support and maintain a patient's jaw in the desired position. Upper notch 12 is sized and configured to receive and engage a portion of the upper central incisors of a patient. Similarly, lower notch 14 is sized and configured to receive and engage a portion of the lower central incisors of a patient as graphically represented in FIG. 5.

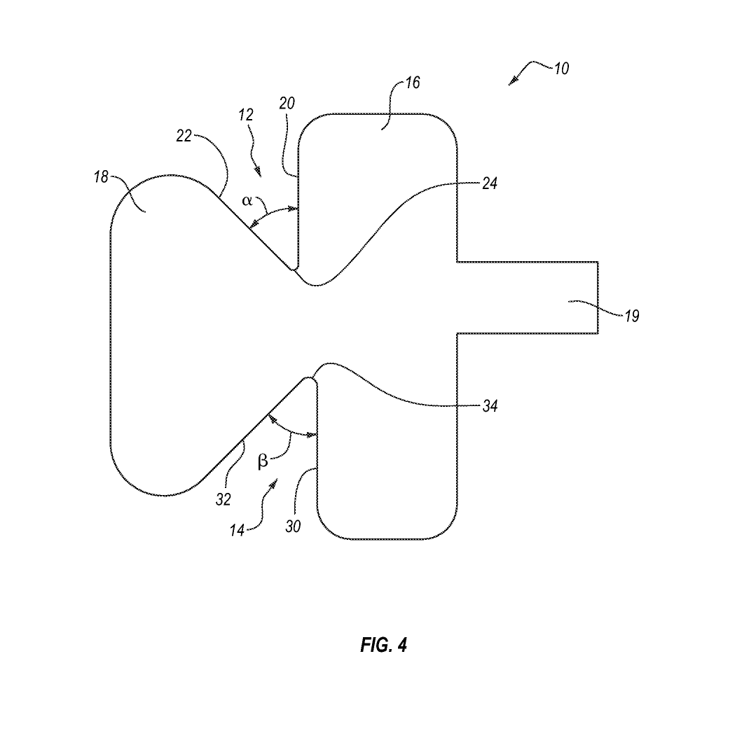

Referring to FIG. 4, upper notch 12 may have a first surface 20 and a second. surface 22, forming an apex 24 at the intersection first surface 20 and second surface 22. First surface 20 and second surface 22 form angle α. As shown in FIG. 4, the shape and depth of upper notch 12 is preferably selected to receive a substantial portion of, and to relatively closely engage the anterior and posterior surfaces of, a portion of the upper central incisors of the patient.

Referring again to FIG. 4, lower notch 14 may have a first surface 30 and a second surface 32, forming a lower apex 34 at the intersection of first surface 30 and second surface 32. First surface 30 and second surface 32 form angle The shape and depth of lower notch 14 is also preferably selected to receive a substantial portion of, and to relatively closely engage the anterior and posterior surfaces of a portion of the lower central incisors of the patient.

In addition, as further illustrated in FIG. 5, upper and lower notches 12 and 14 are preferably sized and shaped so that, when a patient bites down on the bite block 10, the upper central incisors of the patient are gently wedged between the first and second surfaces 20 and 22 of upper notch 12 and the lower central incisors of the patient are gently wedged between first and second surfaces 30 and 32 of lower notch 14 in such a way that the relative positions of the upper jaw and the mandible are securely fixed. In addition, the bite block 10 is securely, and stably, supported by the patient's teeth in such a way that it is not free to rock back and forth or pivot in an anterior or posterior direction relative to the patient's teeth.

In one embodiment intended for adult patients: the depth of upper notch 12 may be in the range of from approximately 5 mm to approximately 10 mm, and may preferably be approximately 6 mm; the angle a may be in the range of from approximately 50 degrees to approximately 55 degrees, and may preferably be approximately 52 degrees; the depth of lower notch 14 may be in the range of from approximately 5 mm to approximately 10 mm, and may preferably be approximately 7 mm; and the angle β may be in the range of from approximately 45 degrees to approximately 50 degrees, and may preferably be approximately 47 degrees.

Referring to FIGS. 3-4, the relative positioning along the longitudinal axis of bite block 10 of first surface 20 of upper notch 12 and first surface 30 of lower notch 14, shown as x in FIG. 3, generally determines the position of the mandible relative of the upper jaw in the horizontal direction, or bite offset. For example, in one embodiment, individual bite blocks 10 may be configured to position the mandible dative to the upper jaw to selectively provide spacing in the horizontal direction, or bite offset, equal to approximately −2 mm, approximately −1 mm, approximately 0, approximately +1 mm, approximately +7 mm, approximately +3 mm, or approximately +mm. As used herein, a “negative” or “−” designation indicates that the patient's lower central incisors are located behind or posterior to the upper central incisors (i.e., in an “overbite” position), and a “positive” or “+” designation indicates that the patient's central incisors are positioned forward of or anterior to the upper central incisors (i.e., in an “underbite” or “advanced” position).

On the other hand, the distance separating the apex 24 of upper notch 12 and the apex 34 of lower notch 14, shown as y in FIG. 3, generally determines the separation between the upper teeth and the lower teeth in the vertical direction, or “bite opening.” For example, in one embodiment, individual bite blocks 10 may be configured to selectively position the mandible relative to the upper jaw to provide spacing in the vertical direction, or bite opening, equal to approximately 4 mm or approximately 6 mm. Thus, the bite block 10 illustrated in FIGS. 2-4 would position the mandible of a patient relative to the upper jaw in an advanced, or forward, position by a distance equal to x in the horizontal plan, and a bite opening in the vertical plane equal to y. In other words, in the illustrated embodiment, the patient's lower central incisors may be located forward of the upper central incisors by a distance of approximately x.

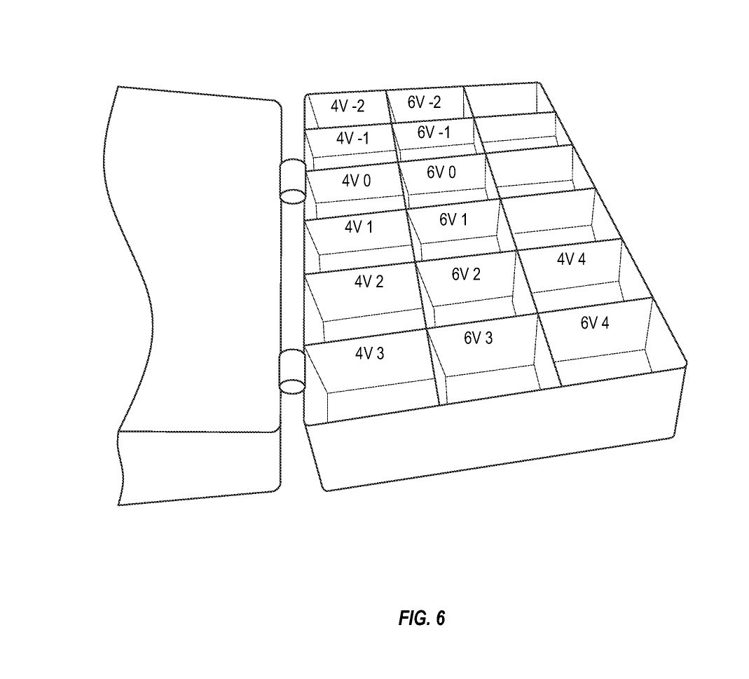

Referring generally to FIG. 6, the invention also contemplates a kit or system, having a plurality of bite blocks 10, with each bite block 10 having a desired configuration for providing a desired combination of (i) a desired mandible advance in the anterior plane and (ii) a desired bite opening in the vertical plane. More specifically, the position of upper notch 12 relative to the position of the lower notch 14 controls the position of the mandible relative to the upper jaw in the horizontal or anterior plane, and can be varied in predetermined increments (e.g., −2 mm, −1 mm, +1 mm, +2 mm, +3 mm, +4 mm, etc.), Similarly, the distance between the apex 24 of upper notch 12 and the apex 34 of lower notch 14 determines the size of the bite opening in the vertical plane, and can be varied in predetermined increments (e.g., 4 mm, 6 mm, etc). Consequently, a separate bite block 10 is provided for each desired combination of (i) mandible position relative to the upper jaw (e.g., −2 mm, −1 min, 0, +1 mm, +2 mm, +3 mm, +4 mm, etc.) and (ii) bite opening (e.g., 4 mm, 6 mm, etc.). For example, a first bite block 10 included in the kit or system may be configured to position a patient's mandible in an advanced, or forward, position of approximately +2 mm relative to the upper jaw and a bite opening equal to approximately 4 mm, a second bite block 10 included in the same kit or system may be configured to position a patient's mandible in an advanced, or forward, position of approximately +4 mm relative to the upper jaw and a bite opening equal to approximately 6 mm, and so on and so forth until all such desired combinations are provided.

In practice, the bite blocks 10 are used to iteratively arrive at the best position and bite opening to optimize airflow and reduce obstructive sleep apnea and/or snoring. This is commonly done using a pharyngometer. The small size of bite block 10 allows a portion of bite block 10 to be positioned or received inside the primary lumen of a pharyngometer during airflow diagnostic testing, and the relatively small thickness of bite block 10 minimizes potential interference with, or restriction of, airflow through the pharyngometer during such testing. In practice, several different jaw positions can be tested, using several different bite blocks 10 to position the jaw in various different positions based on the different configurations of the various bite blocks 10 used, until the best airflow volume is achieved.

Once the best mandible position and bite opening are identified, the corresponding bite block 10 can then be selectively attached to a bite fork so that dental impressions can be made with the patient's jaw positioned by the selected bite block 10 in the position previously determined to provide optimal airflow for the patient. Here, again, the bite block 10 is small enough that it does not significantly interfere with the process of making a dental impression, yet the bite block 10 accurately positions, stably supports, and maintains, the jaw in the desired position (in both the horizontal and vertical directions) while dental impressions are formed. The dental impressions can then be used to fabricate a dental appliance to be worn by the patient for treatment of obstructive sleep apnea and/or snoring.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A bite block for use in diagnostic testing of obstructive sleep apnea and also for use in fabricating dental appliances for treatment of obstructive sleep apnea and/or snoring, the bite block comprising:
a substantially planar body having an upper edge and a lower edge, with an upper notch formed in the upper edge and a lower notch firmed in the lower edge, the upper notch being of a size and a shape to receive a substantial portion of an adult human patient's upper central incisor, the lower notch being of a size and shape to receive a substantial portion of an adult human patient's lower central incisor,
the upper and lower notches being positioned relative one another in a longitudinal direction of the bite block so as to position the patient's upper jaw relative to the patient's lower jaw in a desired position in both horizontal and vertical directions; and
wherein the upper and lower notches are further sized and configured so that, when the bite block is positioned between the patient's teeth and the patient bites down on the bite block, the relative positions of the patient's upper jaw, the patient's lower jaw and the bite block are all securely and stably fixed relative to one another.

2. The bite block of claim 1, wherein the shape of the upper notch approximates the shape of a substantial portion of an upper central incisor of an adult human patient and wherein the shape of the lower notch approximates the shape of a substantial portion of a lower central incisor of an adult human patient.

3. The bite block of claim 2, wherein the upper notch has a first anterior surface, a first posterior surface and a first apex formed at an intersection of the first anterior surface and the first posterior surface, wherein the upper notch has a depth of between approximately 5 mm and approximately 10 mm and wherein the first anterior surface and the first posterior surface form a first angle in a range of from approximately 50 degrees to approximately 55 degrees.

4. The bite block of claim 3, wherein the lower notch has a second anterior surface, a second posterior surface and a second apex formed at an intersection of the second anterior surface and the second posterior surface, wherein the lower notch has a depth of between approximately 5 mm and approximately 10 mm, and wherein the second anterior surface and the second posterior surface form a second angle in a range of from approximately 45 degrees to approximately 50 degrees.

5. The bite block of claim 4, wherein the first apex is offset from the second apex in the longitudinal direction providing a preselected bite offset of the patient's lower jaw relative to the patient's upper jaw.

6. The bite block of claim 5, wherein the horizontal spacing between the first and second apex is selected to provide a bite offset in the horizontal direction equal to one of: approximately −2 mm, approximately −1 mm, approximately 0, approximately +1 mm, approximately +2 mm, approximately +3 mm, or approximately +4 mm.

7. The bite block of claim 4, wherein the vertical distance separating the first apex from the second apex is selected to provide a desired bite opening between a patient's upper and lower teeth.

8. The bite block of claim 7, wherein the vertical spacing between the first and second apex is selected to provide a bite opening in the vertical direction equal to one of approximately 4 mm or approximately 6 mm.

9. The bite block of claim 2, wherein the body has a thickness in a range of approximately 2 mm to approximately 4 mm, wherein the body has an overall length in a range of approximately 20 mm to approximately 30 mm, and where in the body has an overall height in a range of approximately 15 mm to approximately 25 mm. 10, The bite block of claim 1, wherein the bite block is small enough to fit inside a primary lumen of a pharyngometer to facilitate use during diagnostic airflow testing.

11. A set of bite blocks for diagnosing and treating breathing disorders such as obstructive sleep apnea including a presorted set of discrete mandibular positioning elements including positioning means for upper and lower incisors controlling the relative vertical and horizontal position enabling comparative airway measurements;
wherein each bite block within the set provides a different combination of a preselected bite offset and a preselected bite opening, and comprises:
a substantially planar body having an upper edge and a lower edge, with an upper notch formed in the upper edge and a lower notch formed in the lower edge, the upper notch being of a size and a shape to receive a substantial portion of an adult human patient's upper central incisor, the lower notch being of a size and shape to receive a substantial portion of an adult human patient's lower central incisor;
the upper and lower notches being positioned relative to one another in a longitudinal direction of the bite block so as to position the patient's upper jaw relative to the patient's lower jaw in a desired position in both horizontal and vertical directions; and
wherein the upper and lower notches are further sized and configured so that, when the bite block is positioned between the patient's teeth and the patient bites down on the bite block, the relative positions of the patient's upper jaw, the patient's lower jaw and the bite block are all securely and stably fixed relative to one another.

12. A kit for determining and simulating the optimal airway dilation for the treatment of obstructive sleep apnea and/or snoring, comprising a plurality of elements with each element providing a different combination of a preselected bite offset and a preselected bite opening, and each element comprising:
a substantially planar body having an upper edge and a tower edge, with an upper notch formed in the upper edge and a lower notch formed in the tower edge, the upper notch being of a size and a shape to receive a substantial portion of an adult human patient's upper central incisor, the lower notch being of a size and shape to receive a substantial portion of an adult human patient's tower central incisor,
the upper and lower notches being positioned relative to one another in a longitudinal direction of the bite block so as to position the patient's upper jaw relative to the patient's lower jaw in a desired position in both horizontal and vertical directions; and
wherein the upper and lower notches are further sized and configured so that, when the bite block is positioned between the patient's teeth and the patient bites down on the bite block, the relative positions of the patient's upper jaw, the patient's tower jaw and the bite block are all securely and stably fixed relative to one another.
